(19) United States
(12) Patent Application Publication
(10) Pub. No.: US 2004/0180103 A1
(43) Pub. Date: Sep. 16, 2004

(54) Use of plants of the genus hypericum for facilitating the synchronization of a circadian rhythm

(76) Inventors: Gerhard Heldmaier, Marburg (DE); Isabel Klier, Munchen (DE); Tatjana Patzelt, Reichshof-Hespert (DE)

Correspondence Address: Stephan A Pendorf, Pendorff & Cutliff, 5111 Memorial Highway, Tampa, FL, 33634-7356, US

(21) Appl. No.: 10/478,923

(22) PCT Filed: May 24, 2002
(86) PCT No.: PCT/EP02/05730

(30) Foreign Application Priority Data
May 28, 2001 (DE) 10125470.6

(51) Int. Cl. A61K 35/78

(52) U.S. Cl. 424/730

(57) ABSTRACT

The present invention relates to the use of plants of the genus Hypericum or their extracts, respectively, for facilitating synchronization of a circadian rhythm with an external timer, such as in light-dark changes, day-night rhythm changes, time shifts, as occur in the case of long-distance air travel, and the so-called jetlag. The use of extracts from Hypericum perforatum is, however, also suited for use, for example, in cases of forced modifications of the rhythm of activity in persons working shifts. In order to alleviate jetlag symptoms, a dose of 2-200 mg per kg body weight was found to be preferred.

### FIELD OF THE INVENTION

The present invention relates to the use of plants of the genus Hypericum, in particular Hypericum perforatum L., and/or their plant parts and/or their extracts for facilitating synchronization of a circadian rhythm with an external timer.

### BACKGROUND OF THE INVENTION

St.-John's-wort (Hypericum perforatum L.) has been known as a medicinal plant since medieval times.

It was described for the first time by the Greek physician Hippocrates around 460-377 B.C. In 1941, Hypericum was entered into the supplements of the German Materia Medica under the designation “Herba hyperici” and thus was for the first time acknowledged by orthodox medicine.

The use of St.-John's-wort is nowadays known as an alternative for psychopharmacological chemical drugs for the treatment of depressive conditions, for topical treatment of fresh or badly healing wounds, rheumatic complaints, lumbar myalgia, and as a remedy in inflammations of the gastric and intestinal mucosa. The efficacy of St.-John's-wort as an antidepresant has been proven through several clinical double-blind studies, with the mechanism of this efficacy, however, being largely unknown.

Up to now, it has been known that Hypericum extracts inhibit the activity of monoaminooxidase-A and monoaminooxidase-B. Both are enzymes which act to break down serotonin. In addition it is assumed that Hypericum extracts lead to an inhibition of the synaptic re-uptake of serotonin, dopamine and noradrenaline, so that the transmission by these hormones at the synaptic gap is increased. In humans, an increase in nocturnal melatonin production upon ingestion of the extract described (MURCH S. J. et al., 1997: “Melatonin in feverview and other Medicinal plants, Lancet, 350: 1598-1599). What was also described is a prolongation of the deep-sleep phase. Effects of Hypericum extracts on the circadian rhythm of man and animal have previously not been described.

In connection with the disturbance of ciradian rhythms, time shifts owing to long-distance air travel, shift work, working under extreme conditions with artificial sleeping/waking rhythms are prominent.

It is known to administer melatonin as a medicament for more rapid adaptation to time shifts following long-distance air travel, for example. The tryptophan derivative melatonin is produced by the so-called pineal body, a gland projecting in man from the diencephalon into the third ventricle. In the cells of this organ, the so-called pinealocytes, tryptophan from the blood is transformed into melatonin via several biosynthesis steps. Melatonin is approved as a food supplement in the USA, however not as a medicament. In the Federal Republic of German, melatonin preparations are not available on the market.

As far as is known at present, melatonin has the above described effect owing to the following mechanistic attempts at explanation: Melatonin circulating in the blood circulation mainly acts on central, high-affinity binding sites in certain areas of the hypothalamus and of the adenohypophysis.

As far as can be said, serum concentrations of melatonin in humans exhibit a circadian pattern, with high concentrations at night and low ones during the day. This pattern is generated by sympathetic innervation of the pineal body. The daily light-dark change is perceived via the retina in mammals. This information is then neuronally conveyed via the retino-hypothalamic tract to the suprachiasmatic nucleus (SCN), the so-called circadian clock. From there, following further switching locations, post-ganglionic sympathetic fibers originating in the superior cervical ganglion (SCG) reach the pineal body. During the dark phase, these fibers are stimulated by the activity of the SCN, resulting in a release of the neurotransmitter noradrenaline on the nerve endings located in the pineal body. Noradrenaline interacts with adrenergenic predominantly receptors on the membrane of pinealocytes and stimulates the activity of N-acetyltransferase via the cAMP second-messenger system. In accordance with current knowledge, this reaction constitutes the step that determines the speed of melatonin synthesis.

Light acts on the above described melatonin system as a timer which synchronizes the circadian rhythm. In the event of light exposition a cessation of noradrenaline release takes place. From this results an inhibition of melatonin synthesis, which then is followed by a dropping melatonin concentration in the blood.

In humans, melatonin has a serum half-life of about 35 to 50 minutes.

Melatonin thus acts as an endocrine signal for the length of the night, for the duration of the nocturnal melatonin release is proportional to the length of the dark phase. Thus the the melatonin profile enables the organism to determine the respective time of day and season it is in. In man, who has less pronounced seasonal adaptations than animals, melatonin plays a part in the regulation of the sleeping/waking cycle, for which reason melatonin is successfully employed for alleviating adaptation following long-distance air travel, also in the case of so-called jetlag (ARENDT J., ALDHOUS M., MARKS M. (1987): Some effects of jetlag and their treatment by melatonin; Ergonomics 30: 1393-1397 (1987); ARENDT J., ALDHOUS M., MARKS V. (1986): Alleviation of jet-lag by melatonin: preliminary results of controlled double-blind test, BMJ 292: 1170.

### SUMMARY OF THE INVENTION

Accordingly it is an object of the present invention to provide a possible alternative for melatonin as a means of adaptation to an external timer.

The above object is attained by a use of plants of the genus Hypericum and/or their plant parts and/or their extracts, for facilitating synchronization of a circadian rhythm with an external timer, in accordance with claim 1.

As a plant preferred in use, Hypericum perforatum has been found.

Particularly preferred is a use employing Hypericum in the form of plant parts and/or extracts, in particular ethanolic-aqueous extracts and/or CO₂ extracts and/or fractions produced from extracts, in liquid or dried form.

Preferably the external timer encompasses light-dark changes; day-night rhythm; time shifts, in particular following long-distance air travel, preferably jetlag; social timers, in particular forced changes of the rhythmicity of activity in shift work.

A particularly preferred dose for reducing the symptoms of jetlags has been found to be the administration of approx. 2 mg to 200 mg per kg of body weight of Hypericum perforatum dry extracts, with a particularly preferred dose lying in the ranges of 20 to 200 mg/kg, 50 to 200 mg/kg and preferably 100 to 200 mg/kg of body weight.

The efficacy of St.-John's-wort (Hypericum perforatum) was proven in the framework of use in accordance with the invention for facilitating synchronization of a circadian rhythm with an external timer, on the animal model on the one hand, and on volunteer test persons on the other hand.

For investigations into the circadian rhythmicity, the animal model of the Djungarian hamster Phodopus sungorus [a.k.a. Siberian hamster] was found to be particularly well suited, for this is a species exhibiting a pronounced photoperiodical reaction. Its natural habitat extends across the Eurasian steppe areas of the Palearctic from Kazakhstan to Northern China. Under natural conditions, these hamsters already in autumn have a whole number of seasonal adaptations. The entire range of these winter adaptations may be triggered artificially by prolonging the dark phase, placing the emphasis on the central importance of the photoperiod. Phodopus sungorus is an excellent animal model for testing jetlag phenomena, for this species reacts with extraordinary sensitivity to phase shifts brought about by shifting the light-dark rhythm. These animals are virtually incapable of adapting to major time shifts simulated by 5-hour shifts of an artificial daybreak by turning on light. The normal 24-hour rhythms of the animals collapse entirely in the event of such a phase shift.

If the animals are administered Hypericum, on the other hand, preferably in the form of an extract, all of the hamsters could adapt to the new rhythm after 3 to 4 days, and after the adaptation again exhibited a significant 24-hour rhythm.

Use in accordance with the invention was also tested on volunteer human test persons in long-distance air travel from the Federal Republic of Germany to the USA and Japan. It was subjectively reported that all of the test persons accomplished the time shift from −9 h (Los Angeles) to +8 h (Tokio) within 3 days, which found its expression in sleeping/waking rhythms adapted to the new time zone.

### BRIEF DESCRIPTION OF THE DRAWINGS

Further advantages and features of the present invention result from the description of embodiments and by reference to the drawings, wherein:

FIG. 1 shows the results of a control test without Hypericum in a double-plot representation of the locomotoric activity of a Djungarian hamster (Animal #3, male) and periodogram analyses during various test phases. Respective dark phases are marked by the black vertical lines in the double plot. The black columns indicate the overall activity per 24 h;

FIG. 2 shows the results of a control test without Hypericum in a double-plot representation of the locomotoric activity of a Djungarian hamster (Animal # 7, female) and periodogram analyses during various test phases. Respective dark phases are marked by the black vertical lines in the double plot. The black columns indicate the overall activity per 24 h;

FIG. 3 shows the results of a test illustrating the invention with Hypericum in a double-plot representation of the locomotoric activity of a Djungarian hamster (Animal #5, male) and periodogram analyses during various test phases. Respective dark phases are marked by the black vertical lines in the double plot. The black columns indicate the overall activity per 24 h wieder;

FIG. 4 shows the results of a test illustrating the invention with Hypericum in a double-plot representation of the locomotoric activity of a Djungarian hamster (Animal #8, female) and periodogram analyses during various test phases. Respective dark phases are marked by the black vertical lines in the double plot. The black columns indicate the overall activity per 24 h; and

FIG. 5 shows the results of a test illustrating the invention with Hypericum in a double-plot representation of the locomotoric activity of a Djungarian hamster (Animal #12, female) and periodogram analyses during various test phases. Respective dark phases are marked by the black vertical lines in the double plot. The black columns indicate the overall activity per 24 h.

### DETAILED DESCRIPTION

1. Test Animals and Keeping Conditions

The hamsters used in the tests have been bred for several years at the Institute for Animal Physiology of the Department of Biology of Philipps-Universität Marburg. Breeding was carried out in the artificial long day (LD 16:8 under a constant environmental temperature (23±1.5° C.) and atmospheric humidity 60±10%) in walk-in, windowless rooms with artificial illumination. Breeding couples were composed in Makrolon cages and kept with sawdust litter, wooden nest box, feed (hamster breeding diet, Altromin 7014; 2825 kcal/kg) and water ad libitum. In addition they received one piece of apple per week. Young animals were placed one each into a Makrolon cage on day 21, but for the rest kept under the same conditions as the breeding couples.

For the tests adult hamsters of ages from 3 to 12 months were used exclusively.

2. Determination of Dosage for Hypericum Feeding

Apportioning of doses for the Djungarian hamster was carried out in analogy with predetermined dosages of commercially available preparations in human medicine. Here an intake of approx. 800-900 mg of St.-John's-wort dry extract per person and day is recommended.

Starting out from this quantity, it was initially calculated by theoretical computations involving the basal metabolic rate that at a direct comparison of the basal metabolic rate of man and hamster, the dose would amount to 3 mg of Hypericum dry extract per hamster.

Based on preliminary tests, however, the animals were administered a dose of approx. 6 mg per day per hamster.

The Hypericum extracts were mixed with the feed, while taking into consideration that Phodopus sungorus ingests about 3,6 g/d of feed meal (Altromin 7010) during the long day at approx. 23° C. Thus about 1.66 mg of Hypericum dry extract was added per gram of feed meal.

3. Measurement of Melatonin Metabolites in Urine

For the measurement of the 6-sulfatoxymelatonin content as a main melatonin metabolite, the animals were transferred into urine collection cages wherein urine, feces and feed leftovers are separated from the urine, with the latter collecting in a storage container, which was transferred at three-hour intervals to a fraction collector with the aid of a commercially available peristaltic pump.

At the same time a perfusor pump rinsed the system with distilled water so as to wash urine rests from the system and prevent any temporal carry-over of the samples.

Afterwards centrifugation of the urine was performed at 2500 g for five minutes so as to separate off contaminants like hair, feces, and feed rests.

The urine samples were then aliquoted and frozen at −20° C. for measurement of 6-sulfatoxymelatonin.

6-sulfatoxymelatonin measurement was carried out by means of a radio immunassay described, for example, in STIEGLITZ A. (1995): “Flexibilität saisonaler Anpassungen beim Dsungarischen Hamster (Phodopus sungorus)” und der Hirschmaus (Peromyscus maniculatus): “Die Rolle des Pinealorgans.” Dissertation at the Department of Biology of Philipps-Universität Marburg/Lahn. The results are given in ng 6-sulfatoxymelatonin/ng creatinine.

During the investigations it was found that melatonin secretion is quite different in individual animals, however excretion of 6-sulfatoxymelatonin remained relatively constant in individuals during the control phase. Excretion of the measured melatonin metabolite in the controls on days −62 to −60 and days −6 to −3 (a minus sign means before the test) was between 0.35 and 4.8 ng/ng of creatinine. On the third day following the begin of feeding Hypericum perforatum, 6-sulfatoxymelatonin excretion dropped to values between 0.49 to 2.4 ng Metabolit/ng Creatinin. Excretion remained low during days 5 to 8 of the Hypericum treatment. Maximum values of no more than 2.2 ng of 6-sulfatoxymelatonin/ng creatinin were attained. The lowest mean values were measured on day 5. After that, excretion again rose slightly in some hamsters. In Table 1 the absolute and mean 6-sulfatoxymelatonin daily excretion (ng/ng creatinine) of 6 examined hamsters during the control phase and on treatment days 3 and 5 during Hypericum feeding is represented.

TABLE 1

| | Animal | Animal | Animal | Animal | Animal | Animal | | |
|---|---|---|---|---|---|---|---|---|
| Test phase | #1 | #2 | #3 | #4 | #5 | #6 | Mean | SEM |
| Control | 0.4158 | 0.1439 | 0.3144 | 0.2625 | 0.0863 | 0.4094 | 0.2511 | 0.0836 |
| Day 3 | 0.2628 | 0.0674 | 0.1575 | 0.2568 | 0.0549 | 0.1557 | 0.1592 | 0.0607 |
| Hypericum | | | | | | | | |
| Day 5 | 0.1154 | 0.0535 | 0.1312 | 0.1057 | 0.0391 | 0.2193 | 0.1107 | 0.0309 |
| Hypericum | | | | | | | | |

On day 3 of Hypericum treatment, the overall excretion of the measured melatonin metabolite dropped by an average 40%, on day 5 by 59%. This drop was on both days significant in comparison with the control (significance: p<0.05; Dunett).

The measurements in the melatonin test thus clearly show that the 6-sulfatoxymelatonin excretion of Phodopus sungorus is strongly reduced by administration of Hypericum perforatum. As a mechanistic explanation—without being bound by it—two fundamental mechanisms are conceivable:

a) Melatonin synthesis is reduced, with synthesis already being impaired on day 3, although a complete inhibitory action by Hypericum is only achieved from day 5 following begin of feeding.

b) Metabolization of melatonin is modified, so that it is no more metabolized to 6-sulfatoxymelatonin and detectable in urine. This hypothesis matches the curve line of nocturnal melatonin excretion on day 3 of Hypericum administration. The reaction involving Hypericum might be a process capable of saturation, for which enough substance is not available yet on day 3. Owing to the relatively short half-life of melatonin in the body, the curve then rapidly returns to the level of the control conditions. Possibly enough active principle is only present in the body on day 5 so as to clearly lower melatonin excretion more strongly.

4. Measurement of the Animals' Locomotoric Activity

During the Hypericum test, the so-called locomotoric activity of the hamsters was registered with the aid of passive infrared movement detectors (SA 209, Conrad Elektronik). With these detectors installed in the cage lids, temperature changes may be registered within an angle of 90 degrees. An alarm pulse having a duration of three seconds is generated as soon as an object warmer than its surroundings passes one of 28 zones within the 90-degree field. In the cages used (22×17×15 cm), an event was registered whenever the animal moved along the cage floor by more than 1.1 cm.

Where the animals were tested in a treadmill test, activity was determined with the aid of commercially available plastic treadmills (circumference: 45 cm, diameter: 14 cm) affixed on the rear side of a plexiglass cuvette. The rotary movement of the treadmill was converted by a solenoid switch into a change of voltage which was then passed on to the computer. Registration of activity in 6-minute intervals took place with the aid of an IBM PC via A/D converter card. The channels were subject to permanent cyclic interrogation during the entire measurement interval.

5. Phase Shift Test

In the phase shift test it is examined how well Phodopus sungorus tolerates time shifts of 3 h and 5 h.

Phodopus sungorus is an optimum test animal for phase shift tests, for this species exhibits a very sensitive reaction to such phase shifts. A backward shift of 5 h during light phase irrevocably destroys the circadian rhythm. During the phase shift tests, the animals were on the average fed approx. 5 mg of Hypericum extract per day and compared with controls who were given hamster feed without Hypericum extract.

The experiments were conducted in such a manner that the animals were initially kept in their accustomed day-night rhythm, and the locomotoric activity during 9 days was recorded. During this preliminary period, the days are listed as a count-down, i.e., negative values from −8 to 0 are listed.

Starting from day 1, additional feeding of Hypericum perforatum extract is then begun for the test animals of the Hypericum group for 11 days. From day 12, the light is turned off 3 h later, and again turned on after 12 h. The animals thus experience a time shift as taking place in the case of airplane travel in a western direction, such as a flight from Chicago to Los Angeles. On day 28, the light was then turned off 5 h later than at the beginning of the test, and again turned on after 12 h. This simulated time shift corresponds to a flight, e.g., Munich to New York.

During this phase, as well, the locomotoric activity of the Djungarian hamster was recorded. Moreover the total activity per 24 h was recorded, and a so-called periodogram analysis was performed from which the 24-hour rhythmicity of the animals may be seen.

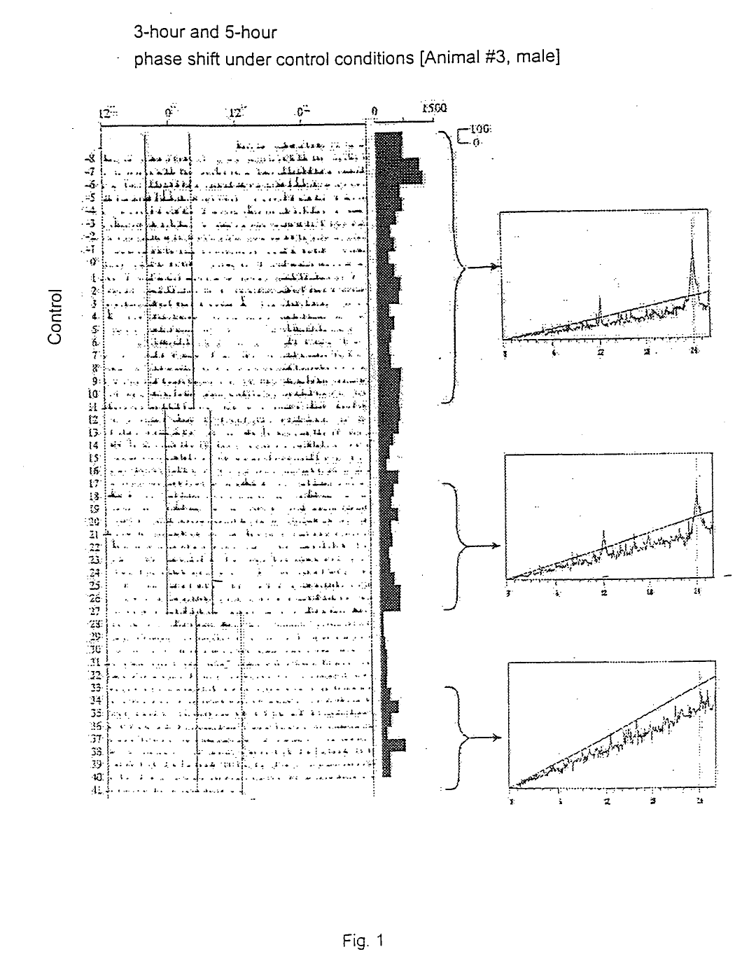

The so-called double-plot representation of the locomotoric activity of the test animals and periodogram analyses, as well as the column diagrams of 24-hour activity are represented in FIGS. 1-5.

As a result of how the test was conducted, each individual animal is its own control animal for locomotoric activity, so that inter-individual fluctuations are not significant.

FIG. 1 shows a double-plot representation of the locomotoric activity of a Djungarian hamster from the control group that was not given any active principle (Animal #3, male), and periodogram analyses during various test phases. Respective dark phases are marked by the black vertical lines in the double plot. The black columns indicate the overall activity per 24 h.

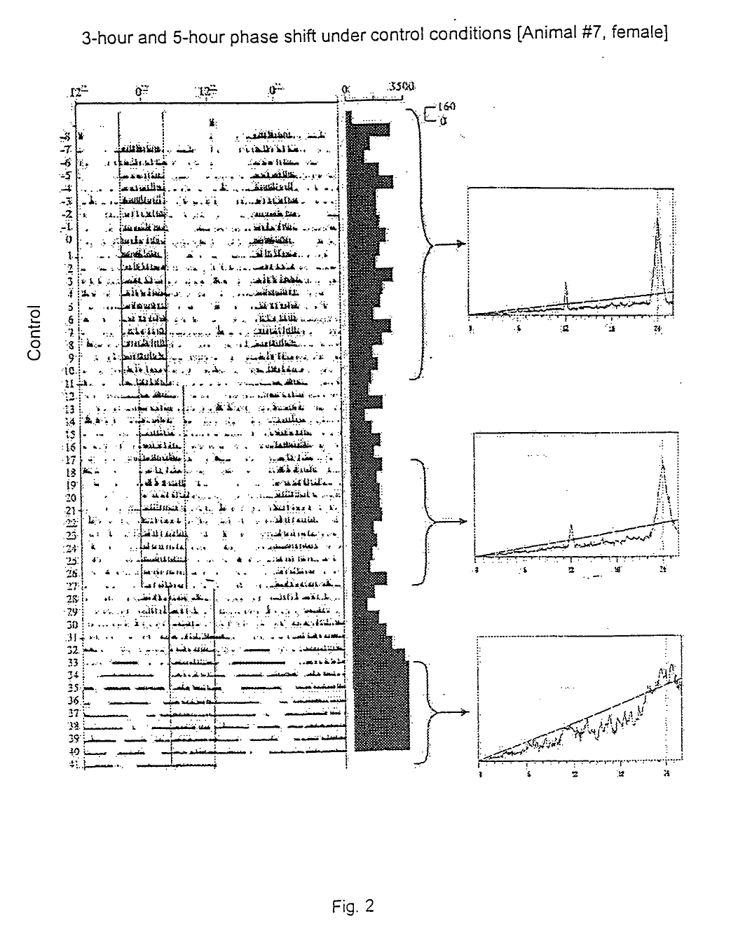

FIG. 2 shows the same double-plot representation of locomotoric activity of a Djungarian hamster from the control group (Animal #7, female), with the representation corresponding to that of FIG. 1.

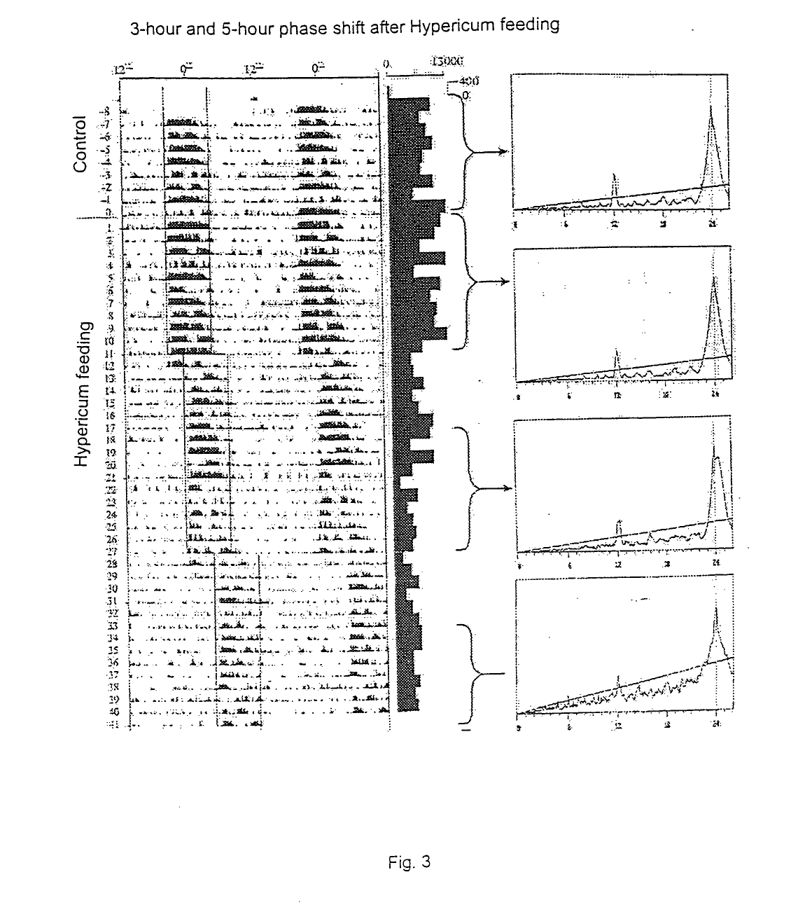

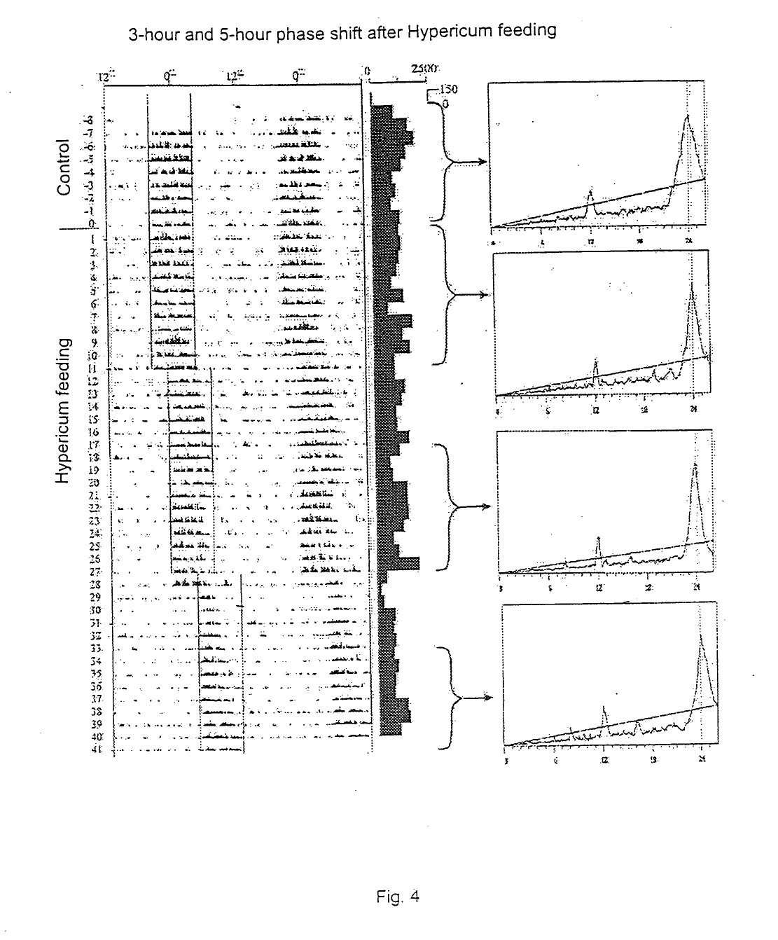

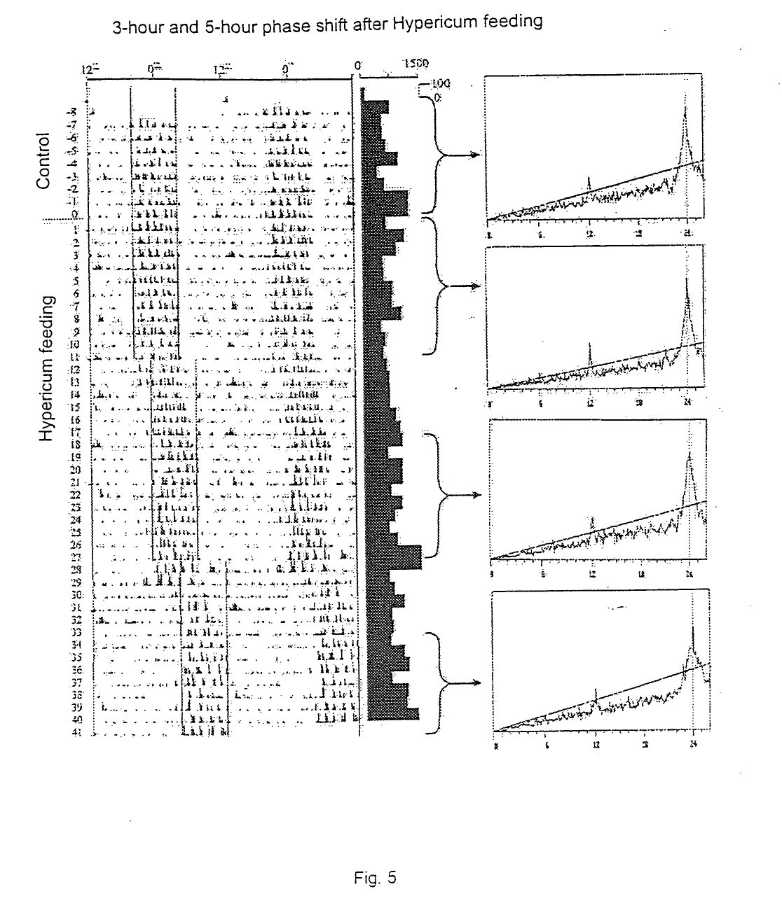

FIGS. 3-5 show double-plot representations of locomotoric activity of a Djungarian hamster, with each animal (FIG. 3: Animal #5, male, FIG. 4: Animal #8, female, and FIG. 5: Animal #12, female), where the animals in the test group were administered about 5 mg Hypericum perforatum extract per animal per os from test day 1.

As may be seen from FIGS. 1 to 5, all of the hamsters follow the first phase shift of 3 h. None of the animals lost its day-night rhythm. This is also documented by the rhythmicity analyses which show that the hamsters again exhibit a significant 24-hour rhythm following adaptation to the new photoperiod after 3 days.

The onset of activity was again adapted to the new light-dark change after 2.8±0.35 days in the case of the control animals and after 3.1±0.3 days in the case of the Hypericum animals. Cessation of activity was only somewhat later adapted to the new light phase, after 6.7±0.8 days in the case of the control animals and after 5.5±0.8 days in the case of the Hypericum animals.

There was no difference between the control group (cf. FIGS. 1 and 2) and the Hypericum group (cf. FIGS. 3 to 5) as regards the speed of adaptation to the new light-dark change.

In the case of the 5-hour phase shift, the control animals did not show any reaction with regard to onset of activity for 1 to 2 days. The circadian rhythmicity of two hamsters had already then been destroyed (cf. FIG. 1, days 29-41). The other 6 hamsters began to shift their activity phase and attained the new light phase within 4.4±0.9 days, measured by the onset of activity. However the circadian rhythm was lost in 5 of these hamsters in the following 10 days. Two of the control animals had already been completely adapted to the new photoperiod, even as regards cessation of activity, before the circadian rhythmicity was destroyed completely.

The periodogram analyses shown in the right-hand column in FIGS. 1-5 of these control animals do not exhibit—following a 5-hour phase shift—any more more significant rhythms in all of these hamsters (cp. lowermost periodogram in FIGS. 1 and 2).

Only a single one of 8 control animals succeeded in adapting to the new light-dark change after 5-hour phase shift. The Hypericum animals (cf. FIGS. 3-5) equally did not exhibit any reaction to the phase shift as regards their onset of activity. Only then a phase shift began, and after an average 3.5±0.5 days, the hamsters became active with the new dark phase. Adaptation of the cessation of activity took longer, on the average 9.1±1.4 days. All in all the individual Hypericum animals showed differences in the rapidity of reacting to the new light-dark change, but all hamsters adapted to the new rhythm within 2 weeks while also being able to continue it. The periodogram analyses represented in FIGS. 3-5 again indicated a significant 24-hour rhythm following adaptation to the 5-hour phase shift. As is shown for the control animals by reference to FIGS. 1 and 2, a backward shift of 5 h during the light phase irrevocably destroys the circadian rhythm of Phodopus sungorus. This has also already been reported in literature (RUBY, N. F. et al. [1996]: “Siberian hamsters free run or became arrhythmic after a phase of delay of the photocycle”, Am J Physiol; 271: R881-890.) When this phase shift occurs under daily administration of melaton, 71% of all animals adapt to the new light-dark cycle (RUBY N. F. et al., [1997]: “melatonin attentuates photic disruption of circadian rhythms in Siberian hamsters”, Am J Physiol; 273: R1540-1549).

The more surprising were the results of the use of Hypericum extracts according to the invention, where the animals adapted better under the influence of Hypericum. For, as melatonin excretion is even lowered under the influence of Hypericum, it rather was to be expected that Hypericum animals would react even more sensitively to a shift of the photoperiod, however the opposite is true.

After these convincing experiments of adaptation by the Djungarian hamsters to external timers while administered Hypericum, an experiment was performed on volunteer test persons.

This was ethically acceptable inasmuch as ready-formulated Hypericum medicaments approved by the government already exist.

10 male test persons, 5 of whom underwent a time shift due to long-distance flight from Munich to Tokyo (+8 h) and 5 additional test persons who underwent a time shift of −9 h (long-distance flight from Munich to Los Angeles), each ingested a dose of approx. 170 mg per kg of body weight in the form of commercially available dragées based on St.-John's-wort dry extract per os for one week prior to undertaking the long-distance flight, during the entire duration of their stay, and one week following their return. All of the test persons had a stay of at least 10 days. Independently of each other, they conformingly rerported an undistiurbed adaptation to the new time zone within 2-3 days upon both arrival and return.

In view of the above, Hypericum perforatum extracts in accordance with the present invention may accordingly be used for synchronizing a circadian rhythm with an external timer, in a particularly preferred manner for light-dark changes of the day-night rhythm owing to the work rhythm, for the treatment of jetlag, and with forced changes of rhythmicity of activity, e.g. in the case of shift work.

What is claimed is:

1. Use of plants of the genus Hypericum and/or their plant parts and/or their extracts for facilitating synchronization of a circadian rhythm with an external timer.

2. Use in accordance with claim 1, characterized in that Hypericum perforatum is used.

3. Use in accordance with claim 1 or 2, characterized in that Hypericum in the form of plant parts and/or extracts, in particular ethanolic-aqueous extracts and/or CO₂ extracts and/or fractions produced from extracts, in liquid or dried form, is used.

4. Use in accordance with any one of claims 1 to 3, characterized in that the external timer encompasses light-dark changes; day-night rhythm changes; time shifts, in particular following long-distance air travel, preferably jetlag; social timers, in particular forced changes of rhythmicity of activity in the case of shift work.

5. Use in accordance with any one of claims 1 to 4, characterized in that Hypericum perforatum dry extract in a dose of approx. 2 mg to 100 mg/kg body weight is administered for alleviating jetlag symptoms.
